(19) United States
(12) Patent Application Publication
(10) Pub. No.: US 2019/0380987 A1
(43) Pub. Date: Dec. 19, 2019

(54) COMPOSITION AND METHOD FOR TREATING MUCOLYTIC AND INFLAMMATORY AIRWAY CONDITIONS

(71) Applicant: Lauranell H. Burch, Durham, NC (US)

(72) Inventor: Lauranell H. Burch, Durham, NC (US)

(21) Appl. No.: 16/008,429

(22) Filed: Jun. 14, 2018

(51) Int. Cl. A61K 31/198; A61K 47/02; A61K 47/12; A61K 47/18; A61K 47/22; A61K 47/26; A61K 9/00; A61P 11/08; A61P 11/12; A23L 33/15; A23L 33/16; A23L 33/175

(52) U.S. Cl. CPC A61K 31/198; A61K 47/02; A61K 47/12; A61K 47/18; A61K 47/22; A23L 33/175; A61K 9/0095; A61P 11/08; A61P 11/12; A23L 33/15; A23L 33/16; A61K 47/26

(57) ABSTRACT

The present invention relates to a novel synergistic combination of N-acetylcysteine (NAC), taurine, a non-thiol anti-oxidant, and magnesium which has been discovered to treat airway inflammation and airway excess mucus.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a mucolytic and anti-inflammatory composition. In particular, it relates to a composition to synergistically reduce mucus and inflammation in a subject's lungs.

### Description of Related Art

There are several diseases of the mammalian lung which result in excessive mucus production and inflammation. There are several compositions which have been utilized in treatment of such conditions.

Cystic Fibrosis (CF) is the most common fatal genetic disease among Caucasians. Although clinical features of CF involve multiple organs, the primary cause of morbidity and mortality is chronic pulmonary infections. The CF gene, Cystic Fibrosis Transporter Regulator (CFTR), is an ATP binding cassette (ABC) transporter that controls transport of multiple molecules responsible for proper hydration and the anti-inflammatory and antimicrobial defense of mucosal surfaces. Loss of CFTR function results in accumulation of copious viscous mucus secretions in ducts and tubules in multiple organs and also at the mucosal surfaces of the respiratory tract where efficient clearance of mucus secretions is important for preventing infection. Loss of CFTR function also results in deficiency of CFTR-transported substrates that are key components of innate immune pathogen defense mechanisms of the respiratory mucosal surfaces. For these reasons, CF is characterized by repeated lung infections and chronic inflammation.

Standard CF therapy relies heavily on repeated use of antibiotics that ultimately fail to eradicate lung infection and lead to damage to beneficial normal bacterial flora and to the emergence of multi-drug resistant pathogens. Decline in lung function among CF patients is seen even in early childhood, and leads to requirement for lung transplant or premature death by respiratory failure.

Defective or insufficient CFTR may be ‘corrected’ (restored to the cell surface) or ‘potentiated’ (increased in activity) by a variety of natural molecules. Alternatively, synthetic molecules may be used. Plant-derived polyphenolic compounds and other natural compounds, particularly dietary compounds, are desirable as CFTR modulating molecules due to lower risk of toxicity. CFTR modulating compounds may be provided by oral administration in an amount and combination effective to cause correction and/or potentiation of CFTR at the tissue site in need of treatment.

Chronic Obstructive Pulmonary Disease (COPD) is a syndrome of chronic airway inflammation, initiated in most cases by smoking or occupational inhalation exposures, which damage the airway surfaces and lung parenchyma over many years. In COPD, accelerated lung function deterioration is accompanied by development of a cough, mucus production and dyspnea, and increasing risk of acute flares of disease, referred to as exacerbations. Exacerbation frequency increases as the disease progresses, further accelerating lung function decline.

The presence of oxidative stress in the airways of smokers and patients with COPD is shown by increased products of lipid peroxidation and altered anti-oxidant status. Like CF patients, patients with COPD are known to have increased numbers of activated neutrophils in their airways that are believed to be attracted to the airways by cytokines IL-8 and TNF-α, which are present in increased levels in the lungs of patients with stable COPD. As CFTR protein resides at the epithelial surface, it is vulnerable to damage by inflammation mediated by toxins and pathogens. In fact, the acquired deficiency of CFTR protein at airway mucosal surfaces is a proposed model for development of COPD.

Oral supplementation with N-acetylcysteine (NAC) has been in use for decades as a treatment for COPD. Some studies that failed to show benefit of NAC for COPD may have used inadequate dosages or study duration to see benefit. Recently, an important clinical study conducted at Stanford University showed that CF patients treated with high-dose NAC (2700 mg of oral NAC per day in divided doses of 900 mg) did not experience lung function decline, while controls declined.

If loss of mucosal surface CFTR function, whether by mutation (CF) or from direct damage of mucosal surfaces (COPD), results in exaggerated inflammatory responses, excessive and sticky mucus, and disease, products that treat both inflammation and mucus production are valuable. In addition, many of the available therapies, e.g. antibiotics, have a deleterious effect on the subject substituting one problem for another.

### BRIEF SUMMARY OF THE INVENTION

The present invention relates to a synergistic combination composition that treats excess mucus and inflammation in the airways of a subject. In particular, the composition contains N-acetylcysteine, which is synergistically enhanced by the addition of additional compounds or compositions.

Accordingly, in one embodiment, there is an oral pharmaceutical composition and administration to a subject for the prophylaxis and treatment of at least one of airway inflammation and airway excess mucus comprising in single or multiple doses:
a) about 1.8 to about 5 grams of N-acetylcysteine per day;
b) about 1 to about 3 grams of taurine per day;
c) about 1 to about 3 grams of a non-thiol anti-oxidant per day; and
d) about 100 to about 750 mg of magnesium per day.

In another embodiment, there is a method of treating at least one of airway inflammation and airway excess mucus comprising administering to a subject in need thereof an oral pharmaceutical composition comprising:
a) about 1.8 to about 5 grams of N-acetylcysteine per day;
b) about 1 to about 3 grams of taurine per day;
c) about 1 to about 3 grams of a non-thiol anti-oxidant per day; and
d) about 100 to about 750 mg of magnesium per day.

### DETAILED DESCRIPTION OF THE INVENTION

While this invention is susceptible to embodiment in many different forms, there is shown in the drawings, if any, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings, if any. This detailed description defines the meaning of the terms used herein and specifically describes embodiments in order for those skilled in the art to practice the invention.

### Definitions

The terms “about” and “essentially” mean ±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

The drawings, if any, featured in the figures, if any, are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

As used herein, the term “oral pharmaceutical composition” refers to a product for the oral administration of a therapeutic composition and/or for prophylactic treatment, treatment of a condition, or prevention of disease composition. In this invention, it is for the condition of airway inflammation and airway excess mucus. Accordingly, the oral pharmaceutical compositions of the invention are formulated in the form of a powder for the purpose of dissolution in an aqueous liquid. Such formulation is well within the art. In one embodiment, the oral pharmaceutical composition powder is sealed in individual dose packets to reduce oxidation. The combination of elements in this composition results in more than the sum of each of the compositions by themselves. In other embodiments, the powder formulation is stirred into food. In one embodiment, the formulation is encapsulated and the capsules are sealed as individual doses in an airtight outer packaging.

As used herein, the term “dissolution in an aqueous liquid” refers to formulating the composition to dissolve, suspend, or the like in an aqueous liquid, e.g. water, orange juice, and the like.

As used herein, the term “administration” refers to addition of the composition to an oral dosage such as an aqueous liquid and dispersing or dissolving it, followed by the subject consuming the liquid.

As used herein, the term “subject” refers to a mammal that has a condition which causes an airway inflammation or airway excess mucus. In one embodiment, the mammal is a human subject.

As used herein, the terms “airway inflammation and airway excess mucus” refer to a condition caused by a condition that causes such inflammation and mucus. In one embodiment, the condition is selected from the group consisting of pulmonary vasculitis, pulmonary sarcoidosis, inflammation and/or infection associated with lung transplantation, acute lung graft rejection or bronchiolitis obliterans syndrome (BOS), pulmonary artery hypertension (PAH), bronchitis, chronic bronchitis, sinusitis, asthma, cystic fibrosis (CF), airways bacterial infection, fungal infection, airways parasite infection, airways viral infection, chronic obstructive pulmonary disease (COPD), persistent pulmonary hypertension of the newborn (PPHN), primary ciliary dyskinesia (PCD), alveolar proteinosis, idiopathic pulmonary fibrosis (IPF), familial pulmonary fibrosis (FPF), eosinophilic pneumonia and eosinophilic bronchitis, acute respiratory distress syndrome (ARDS), mechanical ventilation-associated inflammation and/or infection, ventilator-associated pneumonias, asbestos-related airway disease, dust-related airway disease, silicosis, chemical agent-related airway disease, and any combination thereof.

As used herein, the term “further comprises” refers to additional compositions that can be utilized in combination with the composition of the invention. Such compositions can have anti-mucolytic or mucus reduction qualities as well as anti-inflammatory properties, but other things as well, as needed. In one embodiment, the further composition comprises at least one of the group consisting of niacinamide, potassium, green tea extract, inositol and inositol haxaphosphate, iodine, a sweetener, a flavoring agent, and sodium. Other compositions well known in the art can also be utilized for palatability as well as treatment of other symptoms associated with the disease states being treated. In one embodiment, the sweetener is an alcohol sugar, e.g. xylitol and mannitol.

As used herein, the term “N-acetylcysteine” refers to Acetylcysteine, the N-acetyl derivative of the amino acid L-cysteine, and is a precursor in the formation of the anti-oxidant glutathione in the body. The thiol (sulfhydryl) group confers anti-oxidant effects and is able to reduce free radicals. N-acetylcysteine is soluble in water and alcohol, and practically insoluble in chloroform and ether. When administered orally, in the present invention, the dosage is about 1.8 grams to about 5 grams per day. In other embodiments, it is administered as about 50 mg/kg/day up to about 100 mg/kg/day.

As used herein, the term “taurine” or “2-aminoethanesulfonic acid” refers to an organic compound that is widely distributed in animal tissues. It is a major constituent of bile and can be found in the large intestine, and accounts for up to 0.1% of total human body weight. Dosages in the present invention are about 1 to about 3 grams per day.

As used herein, the term “non-thiol anti-oxidant” refers to sodium ascorbate and ascorbic acid, pH buffers, citric acid, or salt buffers. The non-thiol anti-oxidant synergistically protects the NAC after manufacture. Dosages are about 1 to about 3 grams per day in single or divided doses.

As used herein, the term “magnesium” refers to a cofactor for over 300 enzyme systems that regulate diverse biochemical reactions. Administration is from about 100 to about 750 mg per day. The magnesium is normally utilized as a salt but any form is useful.

In one embodiment, the composition is sealed inside unit dosage packaging in order to reduce oxidation.

As used herein, the term “inflammation” refers to the biological response that occurs in airway conditions caused by either disease or environmental causes. Inflammation is a localized physical condition in which part of the body becomes reddened, swollen, hot, and often painful, especially as a reaction to the airway condition.

As used herein, the term “excess mucus” refers to a slippery viscous fluid produced by the membranes lining the airways of mammals. In the conditions treated herein, “excess” refers to a condition where greater than normal amounts of mucus are produced and especially where that excess causes additional physiological problems.

As used herein, the term “treatment” refers to administering the compositions of the present invention such that they reduce airway inflammation and reduce excess mucus in the airway and includes abrogating, inhibiting, slowing, or reversing the progression of a disease condition, substantially ameliorating clinical or symptoms of a disease condition, and/or preventing the appearance of clinical or symptoms of a disease condition.

As used herein, the term “oral” refers to orally administered compositions of the invention administered to the subject by any suitable oral means. The oral composition is administered as capsules, chewables, tablets, powders, granules, or liquid. In one embodiment, the composition is a powder designed for disolution in a liquid such as water or orange juice and the like.

The invention described herein, when used in the proper composition and dose effective to reduce inflammation, and/or to thin mucus secretions, and/or to modulate CFTR function, is useful to treat airway diseases with an inflammatory component and other diseases of mucosal surfaces with an inflammatory component. It is understood that the compositions of the present invention act in a synergistic manner.

The formulations of this invention may be co-administered with therapeutically active drugs, with other natural extract compounds and supplements, and/or with vitamins or other compounds that may be advantageously utilized in a combination treatment according to the invention. The disclosed compositions may be administered prior to administration of the known therapeutic, for example, at least four hours prior to administration of the known therapeutic. Alternatively, the disclosed compositions may be administered concurrently with the known therapeutic, provided there is no adverse interaction with the known therapeutic agent.

### EXAMPLES

The following composition were prepared and using in the treatment of mucolytic and inflammation of airways.
Serving size: 1 packet dissolved in water, tea, or juice
Suggested use—3 servings per day
Servings per container—30

| | | DELIVER |
|---|---|---|
| | PER | PER |
| Formulation 1 | SERVING | DAY (mg) |
| INGREDIENTS: | (mg) | Per 3 packets |
| Green tea extract (50% polyphenols) | 200 | 600 |
| Inositol | 200 | 600 |
| Inositol hexaphosphate (IP6) | 600 | 1800 |
| Iodide (potassium iodide) | 4 | 12 |
| Magnesium (magnesium taurate, magnesium | 100 | 300 |
| citrate) | | |
| N-acetylcysteine (NAC) | 900 | 2,700 |
| Niacinamide | 50 | 150 |
| Potassium (potassium bicarbonate, | 100 | 300 |
| potassium citrate, potassium iodide) | | |
| Sodium | 250 | 750 |
| Taurine | 666.67 | 2,000 |
| Vitamin C (sodium ascorbate, ascorbic acid) | 600 | 1,800 |
| Xylitol | 4,000 | 12,000 |

Other ingredients: Citric acid, sodium citrate, sodium bicarbonate, natural flavors, natural color, steviol (from Stevia rebaudiana).

### Dosing Regimen

The dosage of the formulation will vary depending on the condition being treated and the state of the subject. Those of a skill in the art will appreciate that the dosing regimen can be varied depending on symptoms, body weight, health and condition of the patient, and the like, and that the optimal dosing regimen can be readily determined using known techniques. The dosing regimen requires either multiple daily administrations for a time limited to duration of the disease or conditions in need of treatment, or multiple daily and/or chronic administrations, particularly among chronic diseases, such as CF, COPD, and primary ciliary dyskinesia. The following results were obtained significantly improving the overall condition of the patient.

Patient 1, cystic fibrosis: Pancreatic enzyme reduction from 25,000 units to 15,000 units, reduction in need for albuterol inhaler from 6 times per day to once per day. Improvement in mucus clearance, color, and consistency. Eliminated morning asthma symptoms and coughing spells associated with gagging and vomiting.

Patient 2, cystic fibrosis: Elimination of nasal and sinus congestion and headaches. Resolution of constipation and bloating. Improved cough clearance. Reduced need for digestive enzymes and weight gain.

Patient 3, cystic fibrosis: Increase in energy and overall well-being. Elimination of muscle aches. No sinus infections in six months. Reduced need for digestive enzymes and weight gain.

### Efficacy Determination

The effect of the treatment may be clinically determined by measurements as described herein. Efficacy may be measured by the reduction of symptoms of inflammation and/or by other endpoints specific to the condition of the diseased subject. For lung diseases, efficacy of a therapy is measured by improvements in pulmonary function tests, improved oxygen saturation, improved quality of life, and reduced frequency of exacerbations, however other endpoints may be desirable to include.

Patients with chronic lung diseases are closely monitored by regular clinic visits. Forced expiratory volume per one second (FEV1) will be measured regularly via spirometry, and also exacerbation rate, and blood O2 saturation. CF quality of life measures (CFQ-R), and exercise capacity are also useful clinical endpoints for lung diseases.

Specifically, treating and/or managing CF can include any one or more of the following: improved lung function, improved quality of life, reduced pulmonary exacerbation, reduced the microbial load, reversion of antibiotic susceptibilities of colonizing pathogens, improvement of the gastrointestinal tract and pancreatic function, and treatment of other mucus membranes of the body such as the sinuses.

Lung function may be improved by increasing the patient's forced expiratory volume in one second (FEV1), the forced vital capacity (FVC), and/or whole-lung mucus clearance. Lung function can be measured by spirometry or plethysmography. Lung function can also be assessed by measuring lung volume according to American Thoracic Society standards as described by the American Thoracic Society.

Pulmonary exacerbation is determined by clinical need for IV antibiotics and/or through presence of the following symptoms: change in sputum volume or color, new or increased hemoptysis, increased cough, increased dyspnea, malaise, fatigue or lethargy, a fever, anorexia or weight loss, sinus pain or tenderness, change in sinus discharge, change in findings on physical examination of the chest, decrease in pulmonary function from a previously recorded value, or radiographic change indicative of pulmonary infection.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art still fall within the scope of the invention as claimed by the applicant.

What is claimed is:

1. An oral pharmaceutical composition and administration to a subject for the prophylaxis and treatment of at least one of airway inflammation and airway excess mucus comprising in single or multiple doses a composition comprising the combination of:
a) about 1.8 to about 5 grams of N-acetylcysteine per day;
b) about 1 to about 3 grams of taurine per day;
c) about 2 to about 3 grams of a non-thiol anti-oxidant per day; and
d) about 100 to about 750 mg of magnesium per day.

2. The oral composition according to claim 1 which further comprises at least one of the group consisting of niacinamide, potassium, green tea extract, inositol and inositol haxaphosphate, iodine, a sweetener, a flavoring agent, and sodium.

3. The composition according to claim 2 wherein the sweetener is an alcohol sugar.

4. The composition according to claim 3 wherein the alcohol sugar is selected from the group consisting of xylitol and mannitol.

5. The oral composition according to claim 1 wherein the at least one of airway inflammation and airway excess mucus is caused by a condition selected from the group consisting of pulmonary vasculitis, pulmonary sarcoidosis, inflammation and/or infection associated with lung transplantation, acute lung graft rejection or bronchiolitis obliterans syndrome (BOS), pulmonary artery hypertension (PAH), bronchitis, chronic bronchitis, sinusitis, asthma, cystic fibrosis (CF), airways bacterial infection, fungal infection, airways parasite infection, airways viral infection, chronic obstructive pulmonary disease (COPD), persistent pulmonary hypertension of the newborn (PPHN), primary ciliary dyskinesia (PCD), alveolar proteinosis, idiopathic pulmonary fibrosis (IPF), familial pulmonary fibrosis (FPF), eosinophilic pneumonia and eosinophilic bronchitis, acute respiratory distress syndrome (ARDS), mechanical ventilation-associated inflammation and/or infection, ventilator-associated pneumonias, asbestos-related airway disease, dust-related airway disease, silicosis, chemical agent-related airway disease, and any combination thereof.

6. The composition according to claim 1 wherein a dosage of the composition is individually packaged in an airtight container.

7. The composition according to claim 1 wherein the composition is in powdered form design to be dissolved or dispersed in a liquid.

8. The composition according to claim 1 wherein the non-thiol anti-oxidant is selected from the group consisting of one or more of sodium ascorbate, ascorbic acid, pH buffers, citric acid, and salt buffers.

9. A method of treating at least one of airway inflammation and airway excess mucus comprising administering to a subject in need thereof an oral pharmaceutical composition comprising:
a) about 1.8 to about 5 grams of N-acetylcysteine per day;
b) about 1 to about 3 grams of taurine per day;
c) about 1 to about 3 grams of a non-thiol anti-oxidant per day; and
d) about 100 to about 750 mg of magnesium per day.

10. The method according to claim 9 which further comprises at least one of the group consisting of niacinamide, potassium, green tea extract, inositol and inositol haxaphosphate, iodine, a sweetener, flavoring agent, and sodium.

11. The method according to claim 10 wherein the sweetener is an alcohol sugar.

12. The method according to claim 11 where in the alcohol sugar is selected from the group consisting of xylitol and mannitol.

13. The method according to claim 9 wherein the at least one of airway inflammation and airway excess mucus is selected from the group consisting of pulmonary vasculitis, pulmonary sarcoidosis, inflammation and/or infection associated with lung transplantation, acute lung graft rejection or bronchiolitis obliterans syndrome (BOS), pulmonary artery hypertension (PAH), bronchitis, chronic bronchitis, sinusitis, asthma, cystic fibrosis, airways bacterial infection, fungal infection, airways parasite infection, airways viral infection, chronic obstructive pulmonary disease (COPD), persistent pulmonary hypertension of the newborn (PPHN), primary ciliary dyskinesia (PCD), alveolar proteinosis, idiopathic pulmonary fibrosis (IPF), familial pulmonary fibrosis (FPF), eosinophilic pneumonia and eosinophilic bronchitis, acute respiratory distress syndrome (ARDS), mechanical ventilation-associated inflammation and/or infection, ventilator-associated pneumonias, asbestos-related airway disease, dust-related airway disease, silicosis, chemical agent-related airway disease, and any combination thereof.

14. The method according to claim 9 wherein a dosage of the composition is individually packaged in an airtight container.

15. The method according to claim 9 wherein the composition is in powdered form design to be dissolved or dispersed in a liquid.

16. The method according to claim 9 wherein the anti-oxidant is selected from the group consisting of one or more of sodium ascorbate, ascorbic acid, pH buffers, citric acid, and salt buffers.
